(19) United States
(12) Plant Patent Application Publication
(10) Pub. No.: US 2003/0115646 P1
(43) Pub. Date: Jun. 19, 2003

(54) Floribunda rose plant named KORpancom

(75) Inventor: Wilhelm Kordes, Sparrieshoop (DE)

Correspondence Address: KLARQUIST SPARKMAN, LLP, One World Trade Center, Suite 1600, 121 S.W. Salmon Street, Portland, OR, 97204, US

(73) Assignee: Jackson & Perkins Wholesale, Inc.

(21) Appl. No.: 10/020,793

(22) Filed: Dec. 14, 2001

(51) Int. Cl. A01H 5/00

(52) U.S. Cl. PLT/148

(57) ABSTRACT

Groundcover rose plant having compact growth; salmon pink flowers; being very floriferous; and resistant to mildew.

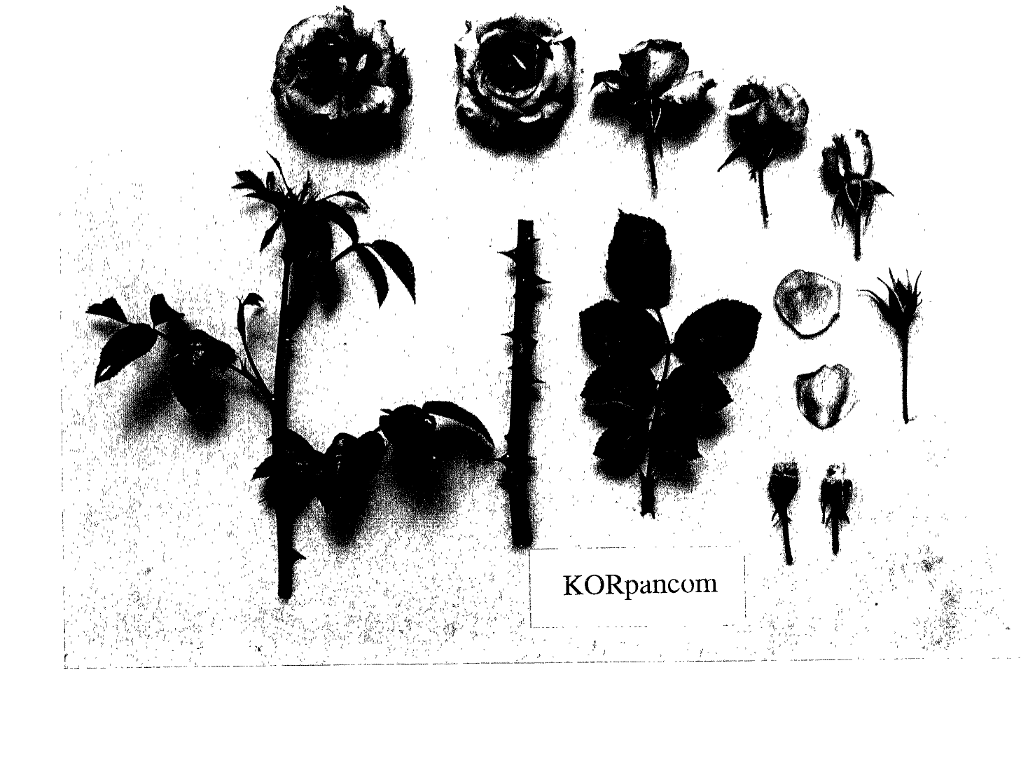

### SUMMARY OF THE INVENTION

The present invention relates to a new and distinct variety of rose plant of the floribunda class which was originated by me by crossing the variety KORwonder (unpatented) with the variety KORgram (unpatented).

### BACKGROUND OF THE INVENTION

The primary objective of this breeding was to produce a new rose variety providing a healthy and low-growing floribunda-type groundcover.

### BRIEF SUMMARY OF THE INVENTION

The objective was substantially achieved, along with other desirable improvements, as evidenced by the following unique combination of characteristics that are outstanding in the new variety and that distinguish it from its parents, as well as from all other varieties of which I am aware:

1. Compact growth;

2. Salmon pink flowers;

3. Very floriferous; and

4. Very resistant to mildew.

Asexual reproduction of this new variety by rooting cuttings as performed at Sparrieshoop, Germany and Somis, Calif., shows that the foregoing and all other characteristics and distinctions come true to form and are established and transmitted through succeeding propagations.

### COMPARISON WITH PARENTS

KORwonder, the seed parent, is a white flowered groundcover with a low, spreading habit. KORgram, the pollen parent, is an upright-growing, pink floribunda. KORpancom is a coral groundcover that is upright and spreading. It is intermediate to the parents in plant habit, has a different flower color to both parents, and smaller flowers that the male parent (KORgram).

### COMPARISON WITH SIMILAR VARIETY

KORpancom has smaller leaves, more compact habit, and more petals than similar varieties KORfullwind and KORtemma. It also has a smaller, more spreading plant and more petals than TANigino and TANotax, other similar varieties. In addition, it is distince from all four in flower color.

### BRIEF DESCRIPTION OF THE ILLUSTRATION

The accompanying illustration shows typical specimens of the vegetative growth and flowers of this new variety in different stages of development, depicted in color as nearly true as it is reasonably possible to make the same in a color illustration of this character.

### DETAILED BOTANICAL DESCRIPTION

The following is a detailed description of my new rose cultivar with color descriptions using terminology in accordance with The Royal Horticultural Society (London) colour chart, except where ordinary dictionary significance of color is indicated.

Parentage:

Seed parent.—KORwonder.

Pollen parent.—KORgram.

Classification:

Botanical.—Rosa hybrida.

Commercial.—Floribunda.

### FLOWER

Observations made from plants of two years of age grown in a garden environment at Somis, Calif. in January, 2000-August, 2001.

Blooming Habit: Continuous.

Bud:

Size.—About 16 to 18 mm long when the petals start to unfurl.

Form.—The bud form is pointed ovoid.

Color.—When sepals first divide, bud color is Red Group 52B. Occasionally petal underside has Yellow-White Group 158D streaks. When half blown, the upper sides of the petals are Red Group 43C; and the lower sides of the petals are Red Group 52B.

Sepals.—Color: Upper surface: Yellow-Green Group 146D with prominent streak of 144D. Lower surface: Green Group 143B with prominent midrib of Yellow-Green Group 145B. Length: 20-22 mm. Width: 4-5 mm. Shape: Subulate. Surface texture: Upper: Hoary. Lower: Margins glandular. There are three lightly appendaged sepals. There are two unappendaged sepals with hairy edges.

Receptacle.—Color: Yellow-Green Group 144B with occassional anthocyanin of Greyed-Orange Group 176B. Shape: Funnel. Size: Small, about 7 mm long×5 mm wide. Surface: Smooth.

Peduncle.—Length: Short, averaging about 25-30 mm. Surface: Glandular. Color: Yellow-Green Group 145B with occassional anthocyanin of Greyed-Orange Group 176A. Strength: Stiff.

Bloom:

Size.—Small. Average open size is 60-65 mm.

Borne.—In pyramidal clusters of 3-5 blooms.

Stems.—Strength: Normal. Average length is about 15-20 cm.

Form.—When first open: High centered. Permanence: Outer petal edges curl slightly.

Petalage.—Number of petals under normal conditions: 30-35.

Color.—The upper sides of the petals are Red Group 52C with a Yellow Group 7D quarter moon spot at base having a 1-2 mm wide Red Group 56D halo. The reverse sides of the petals are Red Group 55A, the base portion of each petal being Red Group 56D. The major color on the upper side is Red Group 52C.

Variegations.—None.

Discoloration.—The general tonality at the end of the first day: Red Group 52C. At the end of the third day: Red Group 52C. By day 5 fades to Red Group 52D.

Fragrance.—Slight.

Petals:

Texture.—Thin.

Shape.—Obovate.

Margin.—Entire.

Apex shape.—Obtuse.

Base shape.—Cuneate.

Form.—Tips slightly recurved.

Arrangement.—Imbricated. Petaloids in the center are few (3-5).

Petaloid color.—Red Group 52C.

Persistence.—Petals drop off cleanly before drying.

Lastingness.—On the plant: Fair (about 7-9 days). As a cut flower: Fair (about 9-11 days).

Reproductive Parts:

Stamens.—Number: 80-100. Anthers: Size: 1 mm. Color: Yellow-Orange Group 17C. Arrangement: Regular around styles. Filaments: Color: Yellow Group 3C. Length: 3-4 mm. Pollen: Color: Greyed-Orange Group 168D.

Pistils.—Number: 40-60. Styles: Color: Yellow-Green Group 154C. Length: 4-5 mm. Stigmas: Color: Greyed-Yellow Group 160B.

Hips.—None observed.

### PLANT

Form.—Bush.

Growth.—Upright, branching.

Mature plant.—Height: 40-45 cm. Width: 55-60 cm.

Foliage: Number of leaflets on normal mid-stem leaves: 5-7.

Size.—70-75 mm long×55-60 mm wide.

Quantity.—Normal.

Color.—New foliage.—Upper side: Greyed-Orange Group 176A with midrib of Yellow-Green Group 145B. Lower side: Greyed-Orange Group 176B. Old foliage: Upper side: Green Group 137B. Lower side: Yellow-Green Group 146B.

Leaflets:

Size.—Length: 35-40 mm. Width: 20-25 mm.

Shape.—Obovate.

Base shape.—Obtuse.

Apex shape.—Acuminate.

Texture.—Upper side: Glossy, smooth.

Edge.—Serrated.

Serrations.—Single, small.

Petiole color.—Yellow-Green Group 145B.

Petiole rachis.—Color: Yellow-Green Group 145B.

Petiole underside.—Prickles.

Auricle shape, if present.—None.

Stipules.—Medium (about 3-5 mm), bearded.

Disease resistance.—Resistant to mildew, susceptible to blackspot and rust under normal growing conditions.

Pest persistance.—Not observed.

Wood:

New wood.—Color: Greyed-Purple Group 183C rapidly becoming Yellow-Green Group 147B. Bark: Smooth.

Old wood.—Color: Green Group 137B. Bark: Smooth.

Prickles:

Quantity.—On main canes from base: Ordinary. On laterals from main canes: Ordinary.

Form.—Straight. Length: 6-7 mm. Width: 5-6 mm.

Color when young.—Greyed-Purple Group 187B.

Color when mature.—Greyed-Orange Group 177B.

Small Prickles:

Quantity.—On main stalks: Few. On laterals: Few.

Color.—Greyed-Purple Group 187B.

Winter Hardiness: Hardy to Zone 5.

I claim:

1. A new and distinct variety of rose plant of the groundcover class, substantially as herein shown and described, characterized particularly by compact growth; salmon pink flowers; being very floriferous, and resistant to mildew.
